(19) United States
(12) Patent Application Publication
(10) Pub. No.: US 2013/0030321 A1
(43) Pub. Date: Jan. 31, 2013

(54) CONCHA ELECTRODE

(71) Applicant: Ming Zhang, Edmonton (CA)

(72) Inventor: Ming Zhang, Edmonton (CA)

(21) Appl. No.: 13/561,647

(22) Filed: Jul. 30, 2012

Related U.S. Application Data
(60) Provisional application No. 61/513,405, filed on Jul. 29, 2011.

(51) Int. Cl. A61B 5/12

(52) U.S. Cl. 600/559

(57) ABSTRACT

A system of monitoring and recording signals generated by ear functions is disclosed. The system may comprise; an electrode having an active portion shaped to fit the concha; a support structure for the electrode for locating the active portion within the concha: and a processing system in electrical communication with the electrode. In a particular embodiment, the support structure comprises a clamp having a first arm and a second arm. in another embodiment, the electrode is on the first arm and a portion of the first arm adjacent the electrode is shaped to fit within the concha, and a second electrode on the second arm serves either as an active electrode combined with the electrode placed on the concha.

### CROSS-REFERENCE TO RELATED APPLICATIONS

This application claims the benefit under 35 USC 119(e) of United States provisional application Ser. No. 61/513405 filed Jul. 29, 2011.

### BACKGROUND

Measurement of both auditory brainstem responses and cochlear microphonic responses is important in the assessment of hearing and hearing loss. Auditory brainstem responses represent the status of the neural components mainly at the brainstem level in the auditory system, while the cochlear microphonic responses represent the status of the functions of the cochlea as they basically arise from the hair cells, i.e., hearing sensory cells. Concha electrodes are interesting because they may be able to accurately assess both auditory brainstem responses and cochlear microphonic responses as well as used to perform electrocochleography.

For reference, a more detailed background may be found in: Zhang M. (2010). Using Concha Electrodes Measuring Cochlear Microphonic Waveforms and Auditory Brainstem Responses. Trends in Amplification, 14(4), 211-217.

### SUMMARY

Disclosed herein is a system of monitoring and recording signals generated by ear functions and auditory system, the system comprising: an electrode having an active portion shaped to fit the concha; a support structure for the electrode for locating the active portion within the concha; and a processing system in electrical communication with the electrode. The support structure may comprise a clamp having a first arm and a second arm. The electrode may receive electrical signals generated by the ear and the auditory system. In some embodiments, the electrode is on the first arm and a portion of the first arm adjacent the electrode is shaped to fit within the concha. In a particular embodiment, the active portion has a convex shape. The clamp may have portions that are respectively divergent and convergent, such that, in operation, the clamp encloses a pinna. The clamp may serve to affix the concha electrode to the concha. In an exemplary embodiment, the clamp is asymmetric such that the first arm extends longer than the second arm. The processing system may comprise a device for recording electrical responses generated by the electrode. In an additional embodiment, the concha electrode further comprises a sound generation system.

Concha electrode means any type of object that is placed at the ear concha position and used for the purpose of recording and measurement of various types of responses. Concha electrode may be used with almost any types of sound delivery devices and methods.

Sound delivery methods may include for example at-ear delivery methods or sound-field delivery methods. Sound delivery devices may include for example supraaural earphones (or also called supra-aural headphones), BAHA (i.e., bone-anchored hearing aids), insert earphone (inside the ear, in-ear headphones, Canalphones), various types of hearing aids, bone-conduction headphone, earphones, or bone conduction oscillators.

The concha electrode includes the method to anchor the concha electrode being used with various types of sound delivery methods and devices.

In a particular embodiment, the clamp may be placed over the pinna such that the electrode on a first arm is located atop the concha. The recording electrode placed at the test ear concha is termed the inverting/active electrode. A second clamp with an identical electrode assembly to the first clamp may be concomitantly placed over the pinna of the contralateral ear such that an electrode on a first arm is located atop the contralateral concha. This electrode may be used as a ground electrode or as a reference electrode. A ground or reference electrode may be placed for example on the forehead. Locations of the reference and ground electrodes can be switched from each other or at other locations on the head or neck. Electrical signals generated by the ear and the auditory system including the brainstem may be monitored via the electrodes, and transmitted through leads to a processing system. A spring mechanism may be included in the clamp to keep the arms in place. In one embodiment, handles may be detachable so that the system may fit under a headphone. The clamp may have holes machined through it for threading electrical leads through the clamp. The electrodes on the first and second arms may be permanently affixed or be readily attached and detached from both the first and second arms. The electrodes may be concave or convex. Various types of convexity or concavity or both may be used.

These and other aspects of the device and method are set out in the claims, which are incorporated here by reference.

### BRIEF DESCRIPTION OF THE FIGURES

Embodiments will now be described with reference to the figures, in which like reference characters denote like elements, by way of example, and in which:

FIG. 1 is a block diagram of an embodiment of the processing system for sound generation and response recording.

FIG. 2a is a diagram of an embodiment of a concha electrode clamp with a first arm and a second arm.

FIG. 2b is a diagram of an embodiment of a concha electrode clamp including a concha electrode.

FIG. 3a is a picture of the system in place in a clinical setting.

FIG. 3b is a line drawing of the system in place on the concha and around the pinna.

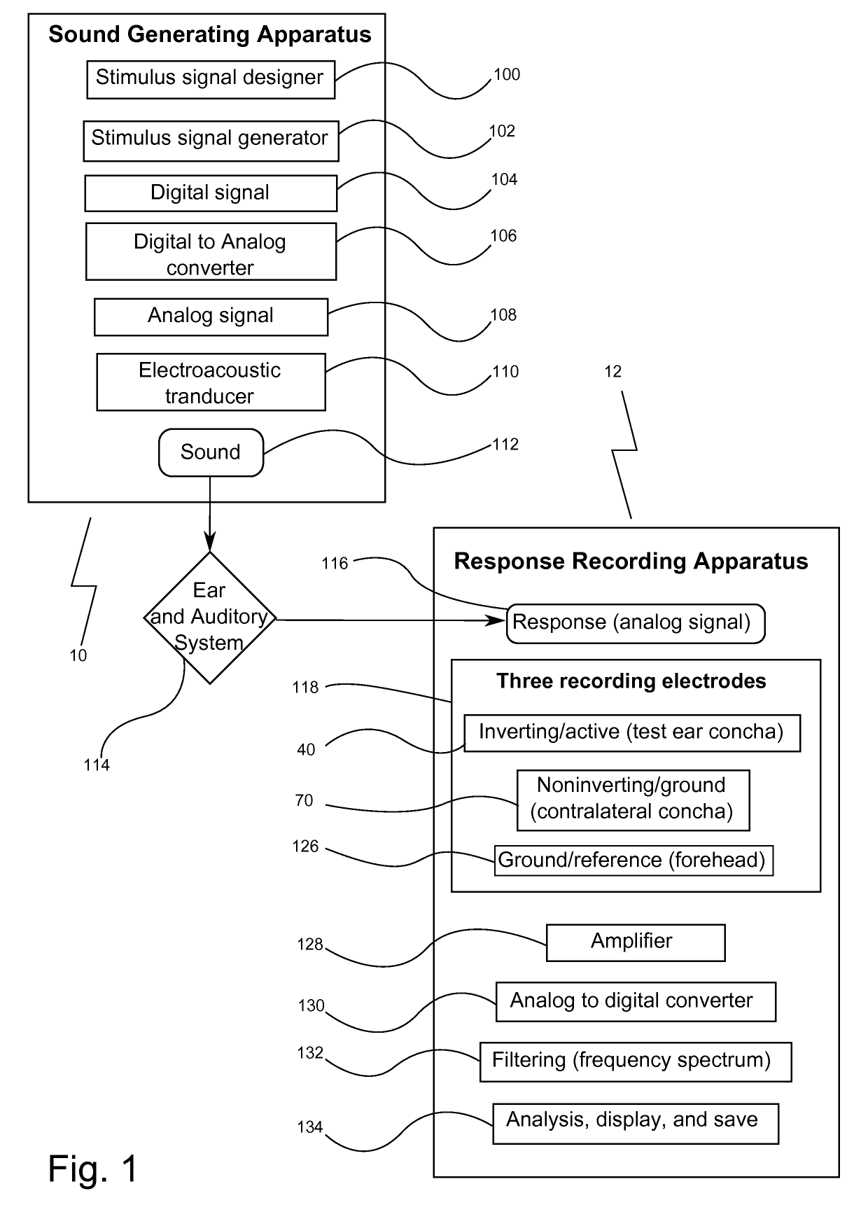

### DETAILED DESCRIPTION

Immaterial modifications may be made to the embodiments described here without departing from what is covered by the claims.

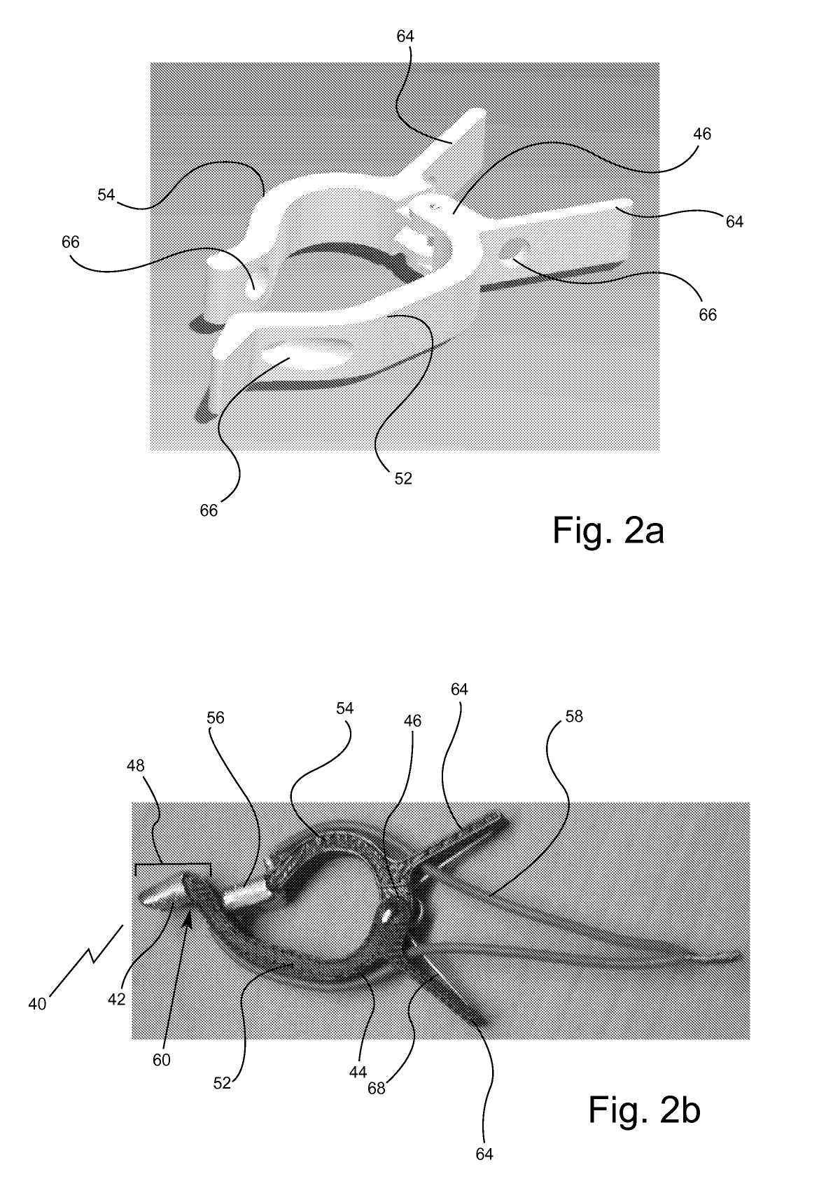

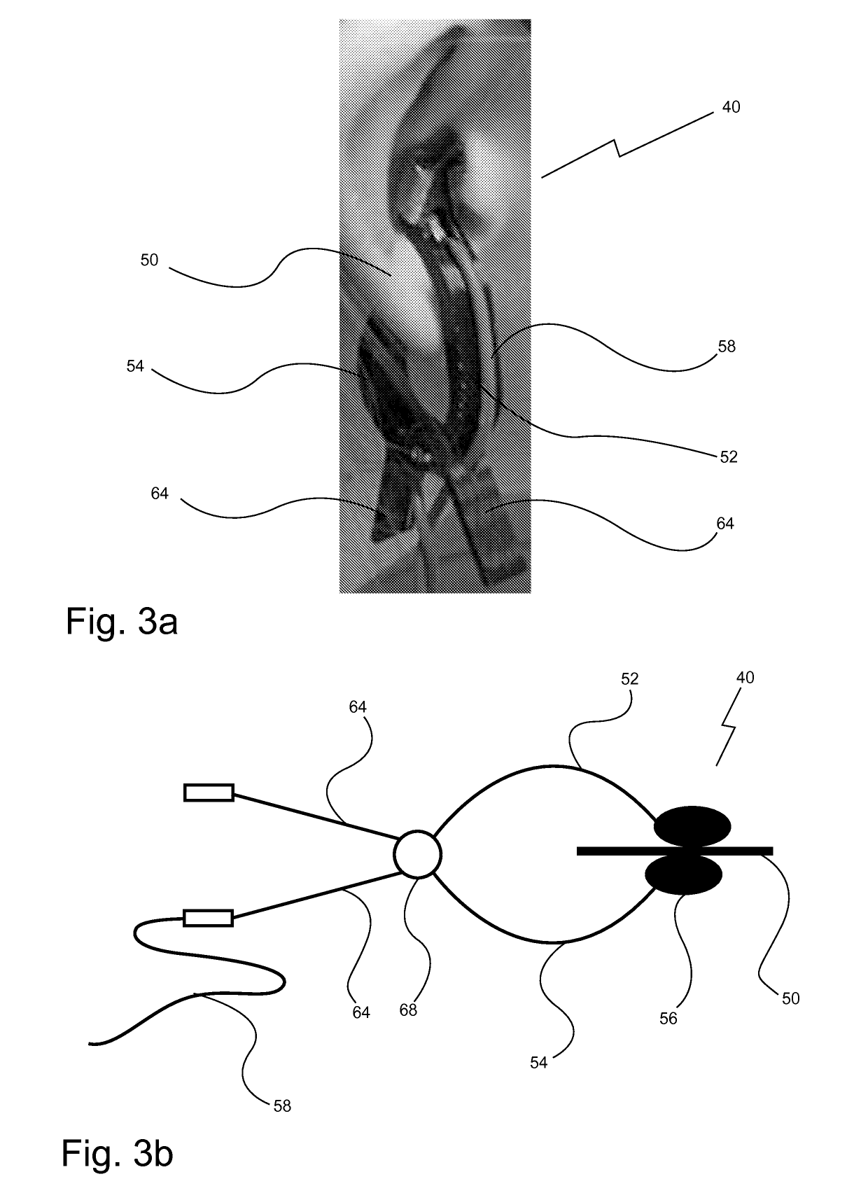

A system of monitoring and recording signals generated by ear functions and auditory system is disclosed herein. The system comprises, as shown in FIGS. 2a and 2b, an electrode 42 shaped to fit the concha; a support structure 44 for the electrode 42 for locating the electrode 42 within the concha; and a processing system in electrical communication with the electrode 42. In a particular embodiment, the support structure 44 comprises a clamp 46 having a first arm 52 and a second arm 54. In some embodiments, the electrode 42 is on a tip 40 of the first arm 52 and a portion 48 of the first arm 52 adjacent the electrode 42 is shaped to fit within the concha. The first arm 52 thus has a curved section for accommodating the pinna, then has a reversal of direction towards the tip 40 on which the electrode 42 is formed. The curvature of the first arm 52 at the portion 48 between the pinna accommodating portion and the electrode 42 is convex and of a suitable size for insertion in the concha. This, together with the shape of the electrode 42, allows the electrode 42 to be located within the concha in use, as shown in FIG. 3a. The electrode 42 may also extend around the portion 48.

In another embodiment, the electrode 42 has a circular section and thus a convex shape suitable for fitting in the concha. In an additional embodiment, the tip 40, including the electrode 42 and adjacent portion 48 of the arm 52 may have a concave shape, which might be obtained for example by rotating the tip 48 including the electrode 42 by 180 degrees about an axis perpendicular to a cross-section through the arm 52. A concave shape is illustrated at 60, but this is not preferred since then the electrode 42 in operation extends too far into the concha. In a further embodiment, the clamp 46 has portions that are respectively divergent and convergent to accommodate a pinna, such that, in operation, the clamp encloses a pinna. The clamp may be asymmetric such that the first arm 52 extends longer than the second arm 54, the longer arm thus incorporating the electrode 42. In another embodiment, the processing system comprises a device 12 for recording electrical responses generated by the electrode. A sound generation system 10 may be included.

In the sound generation apparatus 10 of the processing system, there may be several components. These may include a stimulus signal designer 100, stimulus signal generator 102, a digital signal 104, a digital to analog converter 106, an analog signal 108, an electroacoustic transducer 110, and a sound output 112. The sound is received by the ear 114 and is transformed by the ear and the auditory system into a response 116. In the response recording apparatus 12 of the processing system, several components may be included. The analog response signal 116 may be received through the combination of three recording electrodes 118 using two clamps, one on each ear, and a standard surface contact electrode. The three electrodes include an inverting/active electrode such as electrode 42 placement on the test ear concha, a ground electrode 70, which is electrode 42 belonging to the second clamp 46 placed on the concha of the contralateral ear, and a noninverting/reference ground electrode 126, for example on the forehead. The location of the noninverting/reference and ground electrodes can be switched from each other or at other place on the head or neck. The response recording apparatus may further include an amplifier 128; an analog to digital converter 130; a filtering mechanism 132; and an analysis, displaying and save device 134. The concha electrode can be used by various apparatus made by various manufacturers. Hence, the device comprises a hardware electrode, software signal process (known in the art in itself), hardware electronic circuit to run software (a commercially available processor may be used), interface between the electrode and processor (a commercially available interface may also be used).

In a particular embodiment, the sound generation apparatus 10 generates a sound stimulus, in a digital signal format 102, which may then be converted to an analog signal 104. The analog signal travels through an electroacoustic transducer 108 and may be converted into a sound output stimulus 112. The sound stimulus 112 is received by a human ear 114 and is transformed by the ear and the auditory system into analog electrical signals 116. The analog signals 116 transmit to the concha, and are received by the concha electrode 42. The analog response signal 116 is transmitted to the response recording apparatus 12, and is amplified through an amplifier 128, converted to a digital signal through a analog to digital converter 130, filtered and analyzed through a filtering mechanism 132. The signal may be displayed and saved in a analysis, display and save apparatus 134.

Sound stimuli may be created using the parameters set by the commercially available software. The stimulation system and intensity may be calibrated in accordance with ANSI S3.6-2004 standards. Two types of stimuli may be formulated. One may be a click for the measurement of the auditory brainstem responses. Such a type of click pulse stimulus is most commonly used in the clinic to elicit the auditory brainstem responses. The other may be a tone-burst for measurement of cochlear microphonic responses. The click may be a pulse with width of 100 microseconds which is a wide-band signal. The band may extend up to 10,000 Hz theoretically although a final input to the cochlea in realty is up to around 3-4 kHz due to the effect of modulation of the signal through various media before entering the cochlea. The polarity may be alternatively presented as a typical way to cancel the effect of said polarity on response waveforms.

The recording may also be made through the commercially available systems, and may be calibrated in accordance with ANSI S3.6-2004 standards. The recording may be performed in a sound-treated or sound proofed room, thus the accuracy of threshold measurement with audiometry may be secured. With the suprathreshold recording at an intensity of 80 dB nHL, clear and prominent auditory brainstem responses and cochlear microphonic responses may be recorded. An insert earphone may be used to deliver the acoustic stimulation with delay set at 0.8 ms. The recording electrode 42 (inverting or “−” electrode) may be placed at the concha. The common (not shown, ground or “G” electrode) 126 may be placed at the concha of the contralateral ear and the reference 70 (non-inverting or “+” electrode) may be placed at the forehead or nasion. Compound action potential, wave V, and cochlear microphonics may be recorded from all these three recording electrodes with regular filtering and averages.

The concha electrode may employ a clamp 46, such that pressing the clamp handle 64 opens the two arms 52 and 54 (upper and bottom), and releasing the handle clamps the concha after placing the two arms 52 and 54 on the front and back side of the concha respectively, as shown in FIGS. 3a and 3b. The space between the two circularly-shaped arms may provide room for the helix and antihelix of the pinna 50 so that the helix and antihelix are not clamped and the pressure of the arms is applied on the concha so as to decrease the impendence of the electrodes 42 and 56. The leads 58 take collected responses to the recording amplifier. In a particular embodiment, spacing between the first arm 52 (upper or front) and second arm 54 (lower or rear) may be implemented as follows. In particular embodiments, the spacing is critical for the design of the concha electrode. The spacing may be key to accommodating the remaining parts of the pinna 50 which are not the concha and which are not clamped. In some embodiments, with too small a spacing, the first arm 52 and second arm 54 will exert pressure on non-concha parts and affect blood flow, causing an uncomfortable feeling, and may further alternate the impedance of the electrode on the skin. In other embodiments, with too large a spacing, the first arm and second arm will occupy more space which may affect the application of supra-aural headphone and exert pressure on the mastoid skin. Therefore, in an exemplary embodiment, to fit the contours of different individuals, spacings are designed in various patterns, for example triangle oval, symmetrical oval, customized, or other analogous designs.

The electrode may take a variety of forms. For the shapes disclosed below, the electrodes may have a suitable width and contour (e.g. flat or rounded) that is suitable for placement of the electrode at or in the concha.

In one piece designs, with the concha side facing to the front, some of the following shapes may be used for the concha electrode 42, as disclosed presently. In one embodiment, the frontal side of concha is concaved. Many designs of concavity or convexity may be used. The shape of concave 60 may be approximated using a spherical radius. The radius is small in pediatric populations and large in adult populations. In another embodiment, a set of two or three convex shapes are designed to match the contours for children and adults or adolescents. Using a convex embodiment, because convex is opposite to concave, better matching may be achieved. Some merits of the contoured shape may be an easy anchor; little conductive cream needed between electrode and the skin of the concha because of the smaller interval between the skin and electrode which are similar in shape; and low impedance because good contact is achieved between skin and electrode.

The size for child concha is smaller. Therefore in another embodiment, two or three sizes are designed, for example for children, adults, and adolescents.

In another embodiment, with combinations of features such as contours and size, a set of better shapes may be designed, because of variation between subjects. Therefore combination of different contours and sizes may be designed.

In a particular embodiment, the concha electrode may be flexible. For example, an elastic piece may be used, such as any conductive material as in conductive “rubber”, plastic, or other such materials, being flexible and less rigid. This embodiment may change shape to adapt the shape of the contour of the concha. In another embodiment, the electrode may be rigid, such as for example any conductive material: metal, gold, silver, or other conventional metals used for electrodes. In a further embodiment, the concha electrode may be a rigid and elastic mixture, for example an elastic material facing the skin, and a rigid material on top of the elastic piece. In an additional embodiment, the concha electrode may utilize two piece designs, for both sides of the pinna including the side facing to the rear with two electrodes. An electrode 42 can be placed on the first arm and a second electrode 56 can be placed on the second arm. The response from the ear and auditory system can be measured using the electrodes 42 and 56 independently or simultaneously wherein the later scenario the signals collected by 42 and 56 are combined by a processing system in electrical communication with the electrode.

In an exemplary embodiment, the shape may be convex-concave contoured: for the electrode 42 on the front side of the concha, the convex shape 60 described above may be used. However, because the rear side of concha is convex shaped, for the electrode 56 on the rear side the concave shape matches well and may be used instead. In terms of size, size contour, flexibility, and material type, the two piece design may follow the same implementation as the one piece design.

Note that the same term “radius” used above is used to design the concave-contoured piece to match the rear side of the concha or the front side of the concha.

Leads 58 may be included for connection to the concha electrode 42 and 56. In an additional embodiment, to be used with supra-aural headphones, the leads 58 are designed as a sheet style which is between the edge of the headphone and skin but does not affect the seal so that sound is delivered to the ear instead of leaking around the lead.

In a further embodiment, in use with supra-aural headphone, besides including the spacing distance between the two arms as described above, handles 64 are designed to be detachable. That is, once the arms are placed in position, the handles can be detached for accommodating the supra-aural headphone. In another embodiment, when a clamp is used, the space between two arms may be enlarged to avoid pressure on the rest of the pinna to avoid pressure on concha so that concha gets all the pressure from the clamp, although slight touching on the pinna may be fine. That is, the arms 52 and 54 may diverge and converge to enclose the pinna.

In some embodiments, a concha electrode may have a 6-60 mm radius contour, 2-15 mm width.

For the placement of the concha electrode, the skin may be cleaned, so that the impedance may be reduced to less than five kilo-ohms between inverting, non-inverting, and common ground electrodes.

When performing the recoding of cochlear microphonic responses, several key steps may be followed to ensure that the responses were not due to electromagnetic interference or do not contain electromagnetic interference from the path of stimulation components such as the wire and earphone. The whole wire and earphone may be shielded inside a grounded shielding tube and box respectively to block the discharge of electromagnetic interferences to the recording components such as recording electrodes and related wires. The shielding material may be a commercially available product. A negative control may be performed by clamping the sound delivery tube to block the delivery of acoustic stimulation to the ear while earphone coil is still driven by the stimulation current. With the clamp applied, no sound to the ear, no response evoked, and no sinusoidal waveforms may be recorded. The recorded responses may be monitored and inspected to ensure there is latency before the waveforms are recorded. Note that electromagnetic interference does not have latency while responses do. With these key steps, the recorded waveforms may be valid responses instead of electromagnetic interference.

There are several reasons why the location of the concha may be considered for the placement of the recording electrode. The concha is a part of the auricle or pinna, which is enclosed by the antihelix, antitrogus, trogus, and helical crus. The helical crus separates the concha cavity inferiorly from the concha cymba superiorly. The concha looks like a bowl which is next to the ear canal. Compared to the ear canal, the concha is an easier location to place an electrode as a clamper electrode can be applied. Using the concha electrode is much more economical than the ear canal electrode as it is reusable and it is not a gold-foil electrode which is required for an ear canal electrode. The skin at the concha is easier to clean as the concha is exposed to outside and readily accessible. The skin at the concha is also less sensitive so that less irritation will occur to an infant or a young child. Concha electrode has fewer contraindications such as various disorders in the ear canal and middle ear such as inflammation, infection, tumor, etc. In addition, irritation of the ear canal may trigger unexpected coughs through a reflex via various nerves innervated in the ear canal. The cough in babies never provides you with warning. Unexpected coughs may scratch the soft skin in a baby's ear canal against the electrode or ear plug during insertion. Sensitivity and cough reflex are due to richer nerve innervation to the ear canal than to the concha. The ear canal is innervated by Arnold's nerve (auricular branch of vagus nerve, i.e., tenth cranial nerve), Jacobsen's nerve (tympanic branch of glossopharyngeal nerve, i.e., ninth cranial nerve) and minor fibers of the facial nerve, i.e., the seventh cranial nerve.

Measurement of cochlear microphonic responses with the electrode located at the concha is also feasible as the concha is just adjacent to the ear canal. The distance between the electrode placed in the concha and that which is placed in the ear canal is only a few millimeters apart. Such distance may not generate a significant difference in conductivity of the electrical property in regards to the detection of the electrical response signals. As a result, the responses recorded from the electrode placed at the concha are similar to those recorded from the electrode place at the ear canal. There are many potential applications with the electrode place at the concha, especially for the hearing screening for pediatric populations.

An approach where both cochlear and neural conditions are be evaluated simultaneously may be of a better approach. The electrocochleography procedure records the electrical responses generated within the cochlea so as to assess the function of the cochlea, and enhances results of the auditory brainstem responses. Obtaining a recording including assessment of both nerve and cochlear function reduces the need for further testing of either of the two. Furthermore, simultaneous recording is time-efficient as well as cost-efficient. Moreover, the cochlear microphonic response has spectral and temporal characteristics similar to those of evoked otoacoustic emissions. The cochlear microphonic responses and otoacoustic emissions are elicited from the same generator site. The cochlear microphonic responses can be proven to be a valuable tool because its amplitude is not affected as much as by low frequency noise as the otoacoustic emissions. An in-depth review of the literature shows that research is lacking regarding the use of the cochlear microphonic responses, specifically the low-frequency response.

A concha electrode may offer a compromise between mastoid and ear canal electrodes, and may be an ideal location for this purpose. The concha electrode may be suited for use in clinical settings, and offers an alternative to other electrode locations. Anatomically, concha is directly next to the ear canal. Compared to the ear canal electrode, the concha electrode is easy to apply and more economic because it is reusable. Results indicate that there may be no statistically significant difference between wave I amplitudes recorded at the concha versus at the ear canal. However, wave I amplitudes recorded at the concha and ear canal may be greater than those recorded at the mastoid. As for Wave V, the amplitude may be greatest with the mastoid recording, while no significant difference between the responses recorded at the concha versus at the ear canal. Results may support the hypothesis that amplitude is greater as the electrode is placed closer to the response-generator site; for example, the ear canal is closer to the cochlea and auditory nerve while mastoid is closer to the brainstem.

For the measurement of cochlear microphonic responses, results may show amplitude to be statistically smallest with the mastoid recording compared to the ear canal and concha recordings. However, results may show a significant difference may not be found between the concha bowl versus the ear canal recordings, although the amplitudes recorded at the concha may be smaller than those recorded at the ear canal. This indicates that a similar recording can be obtained from either of these two placements.

Therefore, this is important, because the concha may be an alternative electrode location to the ear canal when there may be contraindications to using an ear canal electrode in a case with disorders in the ear canal and/or middle ear.

Results show identifiable cochlear microphonic response waveforms were obtained with the concha bowl placement, rejecting assumptions that a concha bowl electrode placement cannot be used for this measurement.

The concha is located between the mastoid and ear canal. The responses recorded with the concha electrode are slightly different from those recorded with the electrode placed at the mastoid and ear canal, but the difference in responses is not significant between concha and ear canal electrode placement. In fact, the concha is just directly next to the ear canal so that cochlear microphonic waveforms are similar to those recorded from an ear canal electrode. The mastoid electrode does not warrant the recording of robust cochlear microphonic responses. The wave Vs recorded with an ear canal electrode were not as prominent as those recorded with a concha electrode. The wave Vs recorded at the concha may be slightly smaller than those recorded from a mastoid electrode.

The concha electrode uses the concha as a location. Various designs of concha electrode may be used. In each case, the concha electrode 42 will comprise a conducting element, typically metal, that is shaped for suitable placement at or in the concha together with an electrical lead 58 connecting the concha electrode to the processor of the electronics, as seen in FIG. 2b. Holes 66 may be drilled in the arms and the handles to allow routing of the leads 58 from the electrodes. The tip of the inner electrode may be machined thinner to improve access to the ear canal. A spring mechanism 68 may be included (FIG. 2b).

In an exemplary embodiment, the concha electrode is styled as a mechanic clamper, therefore, easier to place than the mastoid electrode which requires glue- or tape-force to anchor the electrode. The concha electrode may also be easier to place than the ear canal electrode because concha is outside the ear canal and concha electrode is clamp-styled. Thus the placement of concha electrode does not require high degree of cooperation, and is appropriate to the pediatric population as they are not readily to cooperate with. The concha electrode shaped as a clamper can be reused, thus being cost efficient. The concha, being between the ear canal and mastoid, may be an appropriate alternative electrode location to assess both the cochlear and neural conditions simultaneously. Therefore, measurable cochlear microphonic responses and robust wave Vs with the placement of concha electrode may make the concha a unique position in simultaneously assessing the cochlear and neural conditions. This may be critical in the assessment of auditory neuropathy.

Both cochlear microphonic response, wave I and wave V may be measurable with the concha electrode. The wave V may be robust and the cochlear microphonic response and wave I may only be slightly smaller than that measured with an ear canal electrode. The cochlear microphonic responses may be prominent although the wave V may be slightly smaller than that measured with a mastoid electrode. The latency of both cochlear microphonic responses and auditory brainstem responses measured with the concha electrode may not be significantly different from that measured with the mastoid or the ear canal electrode. The concha electrode may be easier to place than the ear canal electrode, suitable for children or pediatric subjects. Using a concha electrode, fewer postauricular artifacts may be observed than using a mastoid electrode.

Because wave I disappears sooner than other waves as stimulus level decreases the recording from the electrode placed at the ear canal may help identify wave I. The responses recorded with a concha electrode may not be significantly different than those recorded at the ear canal. Both wave I and wave V can be identified in responses recoded with concha electrode. This placement may be valuable in that repeated purchase of the costly ear-canal electrode is not needed as concha electrode is reusable, and appearance of artifacts collected by the mastoid electrode can be avoided as concha electrode catch fewer artifacts. The recording may become more effective as recording time becomes shorter as fewer artifacts need to be rejected. Additionally, the ear canal electrode may cause cerumen build-up and may record a smaller wave V amplitude than a concha electrode. Therefore, using the concha bowl placement to obtain recordings may be beneficial in most clinics with regular facility settings because it may be cost efficient and may be effective.

In the claims, the word “comprising” is used in its inclusive sense and does not exclude other elements being present. The indefinite articles “a” and “an” before a claim feature do not exclude more than one of the feature being present. Each one of the individual features described here may be used in one or more embodiments and is not, by virtue only of being described here, to be construed as essential to all embodiments as defined by the claims.

The embodiments of the invention in which an exclusive property or privilege is claimed are defined as follows:

1. A system of monitoring and recording signals generated by ear functions and auditory system, the system comprising:
an electrode having an active portion shaped to fit the concha;
a support structure for the electrode for locating the active portion within the concha; and
a processing system in electrical communication with the electrode.

2. The system of claim 1 in which the support structure comprises a clamp having a first arm and a second arm.

3. The system of claim 2 in which the electrode is on the first arm and a portion of the first arm adjacent the electrode is shaped to fit within the concha.

4. The system of claim 3 further comprising a second electrode on the second arm, the second electrode being in electrical communication with the processing system.

5. The system of claim 4 in which the clamp has portions that are respectively divergent and convergent, such that, in operation, the clamp encloses a pinna.

6. The system of claim 5 in which the active portion of the electrode has a convex shape.

7. The system of claim 6 in which the clamp is asymmetric such that the first arm extends longer than the second arm.

8. The system of claim 7 in which the processing system comprises a device for recording electrical responses generated by the electrode.

9. The system of claim 8 further comprising a sound generation system.

10. The system of claim 2 in which the clamp has portions that are respectively divergent and convergent, such that, in operation, the clamp encloses a pinna.

11. The system of claim 1 in which the active portion of the electrode has a convex shape.

12. The system of claim 3 in which the clamp is asymmetric such that the first arm extends longer than the second arm.

13. The system of claim 1 in which the processing system comprises a device for recording electrical responses generated by the electrode.

14. The system of claim 2 in which the processing system comprises a device for recording electrical responses generated by the electrode.

15. The system of claim 3 in which the processing system comprises a device for recording electrical responses generated by the electrode.

16. The system of claim 4 in which the processing system comprises a device for recording electrical responses generated by the electrode.

17. The system of claim 14 further comprising a sound generation system.
